(19) United States
(12) Patent Application Publication
(10) Pub. No.: US 2020/0268564 A1
(43) Pub. Date: Aug. 27, 2020

(54) LOW PARTICULATE SURGICAL SPEAR

(71) Applicant: Coronet Medical Technologies Ltd., Ripon, NY (US)

(72) Inventor: Wayne Korteweg, North Stonington, CT (US)

(21) Appl. No.: 16/066,211

(22) PCT Filed: Jan. 19, 2017
(86) PCT No.: PCT/GB2017/050121

§ 371 Date: Jun. 26, 2018

(30) Foreign Application Priority Data
Feb. 11, 2016 (GB) 1602483.8

(51) Int. Cl. A61F 13/38; A61L 15/24; A61L 15/28; A61L 15/42; A61L 15/26; B29C 44/02; B29C 44/56; B29C 44/58

(52) U.S. Cl. CPC A61F 13/38; A61L 15/24; A61L 15/28; A61F 13/385; B29K 2001/00; A61L 15/26; B29C 44/02; B29C 44/56; B29C 44/58; A61L 15/425

(57) ABSTRACT

There is disclosed a surgical sponge device comprising a sponge portion and a handle portion The sponge portion is formed from an open cell foam material having an external surface. The handle portion comprises a grip at one end thereof that compressively grips mutually opposed external surface regions of the sponge portion an edge of the sponge portion. Substantially all of the external surface of the sponge portion is formed as an uncut external surface, except for at least one predetermined region of the external surface that is located close to the grip, the at least one predetermined region of the external surface being formed as a cut external surface. Because most of the external surface of the sponge portion is uncut, it will be free of unwanted particulate material.

This invention relates to a surgical spear comprising a sponge material with a low particulate count, and to a method of manufacturing such a surgical spear.

### BACKGROUND

Currently, ophthalmic and other surgical sponges are manufactured using cast blocks or sheets of PVA or cellulose sponge as a starting material. In the context of the present application, PVA sponge is a sponge made using polyvinyl alcohol as the base raw material. The blocks or sheets of sponge are typically cut into sheets or sections of an appropriate width and thickness, generally around 7 to 10 mm, and the sheets are then processed and converted into dry compressed sheets of around 1mm in thickness. The compressed sheets are injection moulded to add handles and then cut to form individual surgical sponge spears for use by medical professionals. Such sponges are known, for example, from U.S. Pat. No. 6,711,879, the content of which is incorporated into the present application by reference. Surgical spears are known for moving and positioning delicate corneal tissues and the like during ophthalmic surgery, as well as for absorbing and controlling excess fluid at the surgical site.

However, known surgical sponge spears suffer from the disadvantage that the manufacturing process, which involves cutting the sponge material, results in debris in the form of tiny sponge particles. The lattice-like structure of the sponge material makes it impossible to cut without creating such particles. Moreover, several cutting steps need to be undertaken before and after the sponge is in the compressed state, and this results in sponge particles becoming embedded in the compressed cut surfaces, only to be released when the sponge spear is used during surgery. This is because the sponge will expand when used during surgery to control and remove liquids, and the expansion will open up the lattice structure of any cut edges, thus allowing the embedded particles to escape into the surgical site. This can be particularly problematic in ophthalmic surgery procedures, where the particles can become embedded in a surgical incision, thus causing complications.

The sponge material of U.S. Pat. No. 6,711,879 is washed prior to packaging so as to reduce the amount of particulates present in the sponge after cutting. However, some debris from the cutting process may still remain embedded and has the potential to come loose when the sponge is expanded at the time of use. The sponge material is cut to size and prepared for the handles to be moulded to the sponge, the handles are moulded to the sponge, and the sponge and handles are cut and separated. Rather than packaging and sterilizing the surgical spears at this point, as would have been normal prior to U.S. Pat. No. 6,711,879, the spear is subjected to an additional set of processes, where the spear is wetted to cause expansion and then put through a washing and filtering process. Once the wash is finished, excess water is extracted from the sponge material in an extraction process, and the spear products then go through a chemical treatment rinse which resaturates the sponge material. A further extraction process is undertaken to remove the excess treatment rinse liquids, and the spear products are then loaded onto drying racks for a lengthy drying cycle. Once dry, the spears are loaded into a machine to re-press the expanded sponge material back down to its original pressed thickness (approximately 1 mm). The spears can then be inspected and packaged for sterilization. During the chemical treatment rinse, the handles will also be coated with the chemical treatment liquid, and this can leave a film or residue on the handles which is not desirable.

Although these additional steps reduce particulate, they also add a significant amount of time and man-hours to the manufacturing process, and hence result in increased costs.

An alternative method for manufacturing sponges is known from U.S. Pat. No. 6,852,258 and U.S. Pat. No. 7,229,579. These disclose a method of making a surgical sponge by introducing sponge raw material directly into individual moulds to form an individual sponge tip portion and create a finished single sponge. Additionally, while the sponge material is in an un-cured state, an individual pre-manufactured handle needs to be precisely placed into the centre of the un-cured material before it is fully cured so as to attach the sponge material to and around the handle. By adding the handle at this point in the process, there is no need to attach handles at a later time. Once the sponge material has gone through its curing cycle, the cured material is removed from the mould and needs to be processed, rinsed, washed, treated, dried, and the sponge pressed flat, down to a thinner thickness to mimic a standard sponge thickness of approximately 1 mm.

During the manufacturing process, especially with PVA sponge, harsh chemicals like formaldehyde and sulfuric acid are used in the making of the sponge material itself. After casting and curing the sponge, the chemicals must be removed before the product is safe for medical use. In this case, the washing process must also include the handle, which will have come into contact the sponge, and therefore become contaminated with these same harsh chemicals. Because the handles are part of the casting process, they also need to be neutralised and tested for chemical and residues.

Although this process produces an individual cast sponge material, which eliminates cutting, the process involves many additional steps and therefore results in a longer and more expensive overall manufacturing process.

In the standard methods of producing a PVA sponge, the sponge material is washed, neutralized and processed to remove all chemicals, cut into sheets, treated and dried for future production into spears. Therefore, the harsh chemical or treatment rinse never comes in contact with the handle portion on the spear.

Casting the individual sponge tips according to U.S. Pat. No. 6,852,258 and U.S. Pat. No. 7,229,579 also gives rise to several functional problems during use of the product by surgeons. Firstly, the way the sponge is cast onto the handle means that the triangle shape of a standard spear cannot be achieved. The sponge ends up facing in the opposite direction to that of a standard spear, therefore altering its shape, look and performance. This is shown in FIG. 1, which illustrates a surgical spear 100 of the type disclosed in U.S. Pat. No. 6,852,258 and U.S. Pat. No. 7,229,579 in both pressed and expanded configurations. Rather than the spear 100 having a triangular pointed shape, this product has a flat and square-ended tip 101, both when wet and in its pressed form. This is because the plastic handle 102 inserted into the sponge 103 has a flat paddle-like form 104 that is surrounded by the sponge material 103, meaning the sponge material 103 can only be pressed flat in the same plane as the plastic paddle 104, and not into the typical standard triangle shape. This pointed shape has been the standard shape of surgical spears since they were first introduced in the 1970s. On a standard spear, the sponge is attached the handle by external means, which pinches the compressed sponge and holds it down during use and expansion. This allows the tip of the sponge to expand and fan out from its base to create a paint brush like shape. The plastic paddle 104 inside the sponge 103 of U.S. Pat. No. 685228 and U.S. Pat. No. 7,229,579 also acts to stiffen the sponge material 103 itself. It is not possible to attach a handle 102 to the sponge material 103 in this process without a paddle-shaped form 104 inside the sponge 103. However, unlike standard spears, which have no hard plastic centre incorporated inside the sponge, this had caused surgeons to be cautious and to use this spear differently. Being aware of a ridged plastic component inside the soft sponge, surgeons must avoid using the product at certain angles, or applying pressure to the surface of the eye which could cause damage.

### BRIEF SUMMARY OF THE DISCLOSURE

Viewed from a first aspect, there is provided a surgical sponge device comprising a sponge portion and a handle portion, wherein the sponge portion is formed from an open cell foam material having an external surface, wherein the handle portion comprises a grip at one end thereof that compressively grips mutually opposed external surface regions of the sponge portion an edge of the sponge portion, and wherein substantially all of the external surface of the sponge portion is formed as an uncut external surface, except for at least one predetermined region of the external surface that is located close to the grip, the at least one predetermined region of the external surface being formed as a cut external surface.

The open cell foam material of the sponge portion is formed by a casting process in a mould. The open cell foam material may comprise a polyvinyl alcohol-based sponge material (PVA sponge), or a cellulose-based sponge, or possibly a polyurethane or other biocompatible sponge material. In all embodiments, it is important that the uncut external surface portions of the sponge portion correspond to external surfaces of an open cell foam material that has been formed in a casting process in a mould, the uncut external surface portions being formed at the interface between an interior of the mould and the liquid material from which the foam material is cast.

The uncut external surface comprises a substantial majority of the external surface of the sponge portion. It is particularly preferred that the uncut external surface comprises the parts of the sponge portion that are distal from the handle and which will typically contact a patient during use. The uncut external surface is porous, so as to allow ingress of fluid. In embodiments where the sponge portion is made of PVA sponge, the uncut external surface portions may be less porous than the open cell structure of the main body of the PVA foam material forming the sponge portion and less porous than the cut external surface portions. The uncut external surface may have a lower open area ratio than an exposed cut surface of the open cell foam material.

It is to be emphasised that uncut external surface portions of the sponge portion will be substantially free of undesirable particulate matter, since these surface portions are defined by a casting process, not by a cutting process.

However, at least one external surface portion of the sponge close to the grip of the handle is formed as a cut surface. This allows a plurality of sponge portions to be cast simultaneously in a mould, the sponge portions connected to each other at relatively small web regions that can be cut after handles have been fitted, thereby facilitating a much more efficient manufacturing process. Because the cut surface portions are located close to the grip of the handle portion and remote from the parts of the sponge portion that are actually used for absorbing fluids from a surgical site, there is a much reduced risk of unwanted particulates from the cut surface portions contaminating the surgical site.

The sponge portion may have a substantially triangular or triangular prism shape, with an apex of the triangular or triangular prism shape forming a first, pointed end and an opposed base of the triangular or triangular prism shape forming a second end which is gripped by the handle portion is attached.

Alternatively, the sponge portion may have a coffin shape, leaf shape, teardrop shape, rhomboid shape, ovate shape or may be formed as a cone or a ball.

It is important to note that, in preferred embodiments, the handle portion does not extend inside the volume of the sponge portion, and the sponge portion in use will therefore be soft and pliable and much less likely to cause damage to the site of surgery than a surgical spear in which a rigid or semi-rigid handle portion is incorporated into the inside of the sponge portion during casting of the sponge portion.

Viewed from a second aspect, there is provided a sponge web formed from an open cell foam material, the sponge web comprising a plurality of individual sponge portions each having a base edge and an opposed tip region, the sponge web having an uncut external surface, and adjacent sponge portions being connected to each other in the web at their base edges.

The plurality of individual sponge portions may comprise first and second rows of facing sponge portions arranged such that the respective tip regions of the first and second rows interdigitate but are spaced from each other by a gap.

The sponge web may further comprise a plurality of handle portions each having a grip at one end thereof, the grip of each handle portion being attached to the base edge of an individual sponge portion.

The sponge web may be formed from a polyvinyl alcohol-based (PVA) sponge material, a cellulose-based material or other suitable materials that can be cast in a mould.

Because the external surface of the sponge web is uncut, it will be substantially free of particulate matter resulting from cutting the sponge web. While some trimming of the base edges may be required, and although it is necessary to separate the sponge portions from each other at their base edges by a cutting process, the resulting cut edges and any associated particulate material will be remote from tip regions of the sponge portions, which will be in contact with a surgical site during use of the surgical spears thus formed.

Viewed from a third aspect, there is provided a method of manufacturing a surgical sponge device comprising a sponge portion and a handle portion, the method including the steps of:

i) providing a mould configured to define a plurality of voids that are connected to each other only along at least one edge portion of the mould;

ii) filling the mould with reagents selected to generate an open cell foam material within the mould, the open cell foam material taking the form of a plurality of sponge portions having shapes defined by the plurality of voids, the sponge portions connected to each other only at base regions thereof corresponding to the void connections in the mould, the open cell foam material having an uncut external surface where it contacts the mould;

iii) removing the open cell foam material from the mould;

iv) washing and treating the open cell foam material;

v) subsequent to step vi); connecting multiple handle portions to the open cell foam material at the base regions of the sponge portions and cutting the open cell foam material to separate the open cell foam material into a plurality of surgical sponge devices, each device having a handle portion connected to a sponge portion formed of the open cell foam material having the uncut external surface formed substantially all of its external surface except for at least one predetermined region of the external surface that is located close to the point of connection of the handle portion, the at least one predetermined region of the external surface being formed as a cut external surface, wherein the handle portions each comprise a grip at one end thereof that mechanically grips mutually opposed external surface regions of the sponge portion an edge of the sponge portion.

The sponge material may be a polyvinyl alcohol-based sponge material (PVA sponge), or a cellulose-based sponge, or possibly a polyurethane or other biocompatible sponge material. The reagents required to form such sponge materials in a mould are known to those of ordinary skill in the art. Generally, the reagents comprise a curable liquid material (comprising, for example, PVA or cellulose or the like) and a pore forming agent. The pore forming agent may be a foaming agent that generates bubbles within the liquid material, or may be a soluble solid material such as starch or salt particles that can be washed away after curing to leave voids or pores in the sponge material.

The mould preferably comprises a single mould cavity that can easily be filled in one step with reagents selected to generate an open cell foam material within the mould.

It will be appreciated that the special technique of using a mould with a plurality of voids that are connected to each other only along an edge portion of the mould results in a web of open cell foam material comprising a plurality of surgical sponge portions that are connected to each other only at regions that are remote from the regions that will form the tips of the finished surgical sponge devices. This means that when the individual sponge portions are cut from the web, the cut surfaces will be small and remote from the working tips, thus reducing the risk of particulate contamination of a surgical site during use.

The mould may have a generally saw-tooth configuration, each void comprising a tooth of the saw-tooth configuration. A web of sponge material formed in such a mould will thus have a complementary saw-tooth configuration, each tooth defining a sponge portion of a surgical sponge device, with a pointed tip that has an uncut external surface due to contact with the interior of the mould. The teeth of the web of sponge material are joined together at base portions remote from the pointed tips. In some embodiments, the tips may be blunt rather than pointed. Instead of a saw-tooth profile, the mould may be configured to provide a sinusoidal or other shaped profile.

In one embodiment, a double-sided mould may be used. The double-sided mould comprises a central wall following a saw-tooth, sinusoidal or other appropriate path, thus defining two sets of complementary, inter-engaging voids. This allows two webs of sponge material to be moulded simultaneously using a single mould, thereby helping to improve production efficiency.

By way of the methods disclosed above, it is possible to form webs of sponge material each comprising a plurality of triangular surgical sponge portions each having an uncut external surface, the triangular surgical sponge portions joined to each other only at adjacent base portions remote from the apexes of the triangular sponge portions.

The mould may define a plurality of voids having shapes other than triangular, for example a coffin shape, leaf shape, teardrop shape, rhomboid shape, ovate shape. In some embodiments, the voids may be formed as cones or balls.

Where inner surfaces of the mould are smooth, the resulting uncut external surface on the web of sponge material will also be smooth. It is possible to provide a textured uncut external surface by using an appropriate textured surface on at least parts of the inner surfaces of the mould. Textured uncut external surfaces may be preferred for some applications.

After removal from the mould, the webs of sponge material can be washed and chemically treated in the conventional manner, with the sponge portions remaining connected to each other at their base portions.

The washed and treated webs may then be dried and pressed or at least partially pressed to a desired thickness, for example to a thickness of around 1.0 to 2.0 mm, for example 1.5 mm. In in some embodiments, it may be preferred to leave the centre unpressed, while pressing the base where handles will be attached to a thickness of, for example, approximately 1.4 mm. The results of this press will leave the triangle sections expanded in the finished product, which is preferred by some surgeons.

Handle portions may then be attached to the base portions of the surgical sponge portions. This may be facilitated by inserting the pressed web into an injection mould. The handle portions may be configured with gripping portions for gripping the base portions of the surgical sponge portions. By attaching the handle portions at this stage, the handle portions are not subjected to the harsh chemicals of the sponge manufacturing process during casting, or to the chemicals used during the stage of chemically treating the web of sponge material after manufacture. This means that it is not necessary to test for and, where necessary, neutralise such chemicals on the handle portions later on, nor to remove possible residue from any final chemical treatment, thus improving manufacturing efficiency.

Once the handle portions have been attached, the individual surgical sponge portions may be cut from the web by cutting through the connecting base portions. Additional cuts may be made to shape the base portions as desired, provided that the cuts are made only in the region of the base portions adjacent to the points of attachment of the handle portions. In some embodiments, all of the individual sponge portions may be cut simultaneously by using a plurality of blades. It will be noted that the amount of cutting is significantly less than would be the case if the sponge portions were cut out of a sheet or strip of sponge material, and also that substantially all of the external surface of each sponge portion that is intended for contact with a surgical site will comprise an uncut external surface as a result of the sponge casting process. This helps to reduce or eliminate particulate contamination by a significant amount.

Once the surgical sponge portions with attached handle portions have been cut from the web, they can be inspected for quality control, sterilised and packaged for subsequent use.

An advantage of embodiments of the present disclosure is that the novel combination of casting a web of sponge material, subsequently affixing handles, and then performing minimal cutting of portions of the sponge material that will not come into contact with a surgical site means that the speed of production is significantly faster and cheaper than attaching individually cast sponge members to separate handles, without significantly increasing the risk of particulate contamination. Furthermore, attaching the handles after the sponge material has been cast and treated means that there is no risk of contamination of the handles with harsh sponge treatment chemicals.

Further advantages will be apparent from the following detailed description.

### BRIEF DESCRIPTION OF THE DRAWINGS

Embodiments of the invention are further described hereinafter with reference to the accompanying drawings, in which:

FIG. 1 shows a prior art surgical spear in pressed and expanded conditions;

FIG. 2 shows a surgical spear of an embodiment of the present disclosure in pressed and expanded conditions;

FIG. 3 shows a sponge web with an uncut external surface;

FIG. 4 shows an alternative sponge web with an uncut external surface;

FIG. 5 shows a mould suitable for casting the sponge web of FIG. 4;

FIG. 6 shows how the sponge web of FIG. 4 can be trimmed;

FIG. 7 shows the addition of handle portions to the edges of the sponge web of FIG. 4;

FIG. 8 shows how the sponge web and attached handles portions of FIG. 7 can be cut into surgical spears having different base shapes;

FIGS. 9 and 10 show examples of shapes for the surgical spears;

FIGS. 11 to 14 show further alternative sponge webs;

FIG. 15 shows the sponge web of FIG. 4 subjected to a partial edge press and to a full press;

FIG. 16 shows the edge pressed sponge web of FIG. 15 provided with handle portions; and

FIG. 17 is a process flow chart illustrating a manufacturing technique.

### DETAILED DESCRIPTION

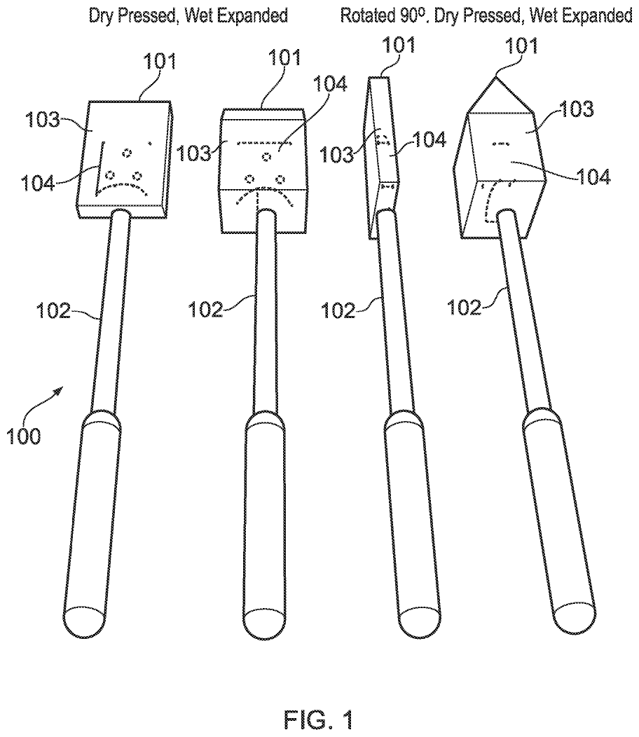

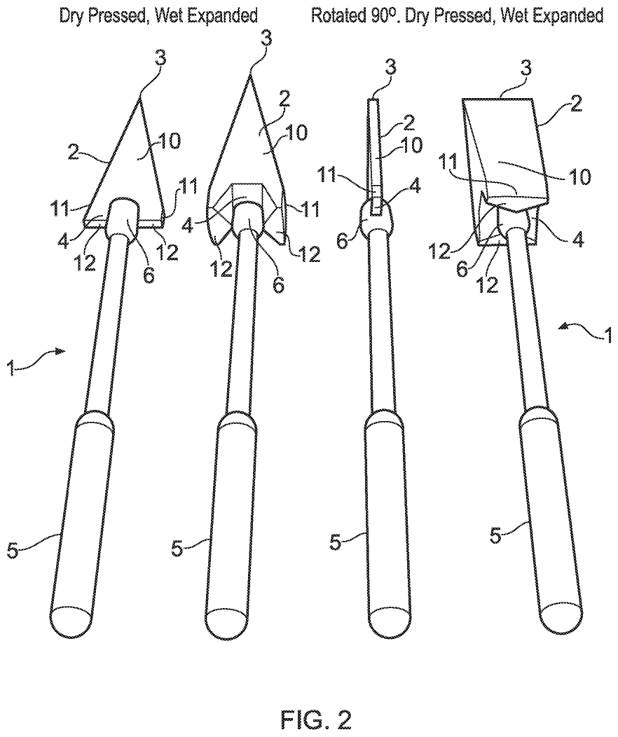

FIG. 2 shows a surgical spear 1 according to an embodiment of the present disclosure in both pressed and expanded conditions, and in two orientations. The spear 1 comprises a triangular surgical sponge portion 2 having an apex 3 and a base portion 4. The sponge portion 2 is made from an open cell foam material such as a cast polyvinyl alcohol (PVA), cellulose, polyurethane or other material having an uncut external surface 10. The spear 1 further comprises a handle portion 5, typically made of a plastic or thermoplastic material, the handle portion 5 including a pair of gripping members 6. The gripping members 6 are configured for gripping the base portion 4 of the surgical sponge portion 2. The only cut external surface portions of the sponge portion 2 are the edge portions 11 and, optionally, the base edge portion 12. It can be seen that the cut external surface portions 11, 12 comprise only a very small part of the overall surface area of the sponge portion 2, and are located close to the gripping members 6 and thus remote from the apex 3 of the sponge portion 2, which is the part of the surgical spear 1 that is used for contacting a surgical site. A substantial majority of the overall surface area of the sponge portion 2 comprises the uncut external surface 10, which is free of particulate. Unlike the prior art spear of FIG. 1, there is no internal paddle-shaped member 104, and the shape of the spear 1 is similar or identical to that of traditional surgical spears preferred by many surgeons.

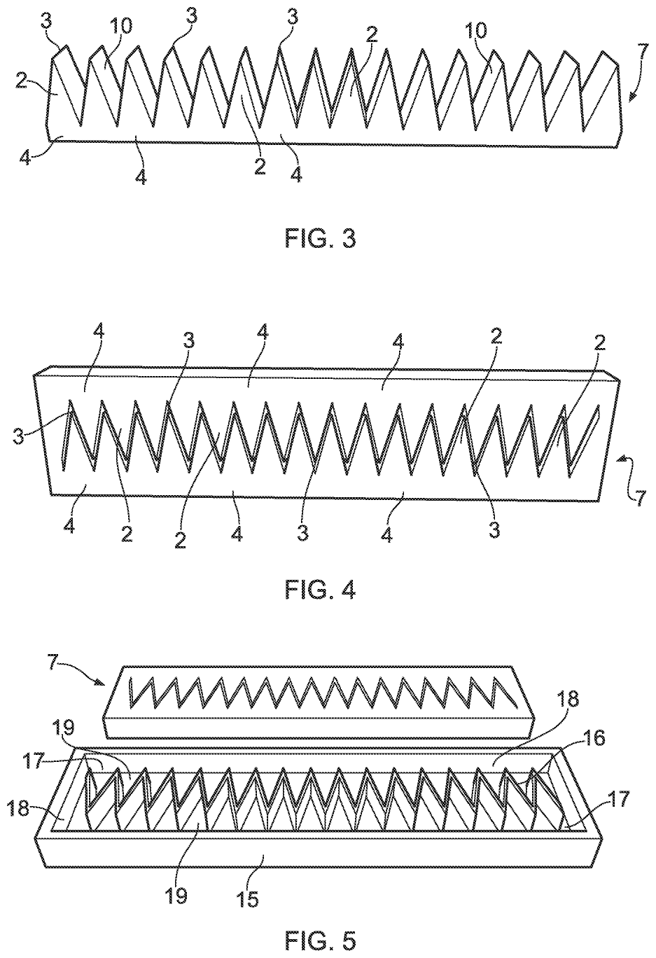

FIG. 3 shows a web 7 of surgical sponge material after it has been cast and cured in a mould of complementary shape, but prior to pressing. The web 7 has a saw-tooth configuration, with individual triangular surgical sponge portions 2 joined together at their base portions 4. Each surgical sponge portion 2 has a pointed apex 3. In the specific embodiment shown in the Figure, the web 7 has a thickness of about 8 mm, and the base portion 4 of each triangular surgical sponge portion 2 is about 8 mm wide. The base portions 4 have a height of about 5 mm for ease of handling during processing. The surface portions of the web 7 that have been in contact with the mould comprise uncut external surface portions 10 which are substantially free of undesirable particulate. The embodiment shown has 16 triangular surgical sponge portions 2 joined together in the web 7, but it will be appreciated that other numbers of sponge portions 2 may be formed in a web 7 as required.

FIG. 4 shows an alternative web 7 of surgical sponge material after is has been cast, cured and removed from a mould. The web 7 is similar to the web 7 of FIG. 3, except that it has been formed in a double-sided mould (see FIG. 5). This allows the simultaneous casting of two sets of inter-engaging triangular surgical sponge portions 2, each set being held together by a respective base portion 4. After pressing and drying, the thickness of the web 7 is around 1.4 mm.

FIG. 5 shows a cut open or cross section of a mould 15 suitable for casting the sponge web 7. The mould 15 includes a central divider 16 that serves to define the saw-tooth configuration of the sponge portions 2. It will be understood that the central divider 16 may take shapes other than a zig-zag shape, depending on the desired shape of the finished surgical spear sponge portions 2. For example, the apexes may be flattened or rounded rather than sharply pointed, or the central divider may have a sinusoidal other configuration as required. A pathway 17 extends around the central divider 16 and inside inner walls 18 of the mould 15 so as to allow the liquid sponge forming components easily to fill the mould 15, and to define the connecting base portions 4 of the sponge web 7. When casting the sponge web 7, surface areas of the sponge web that cure against surfaces of the mould 15, such as the base 19 and a matching top portion (not shown), the inner walls 18 and the central divider 16, will form the uncut external surfaces 10 of the sponge web 7.

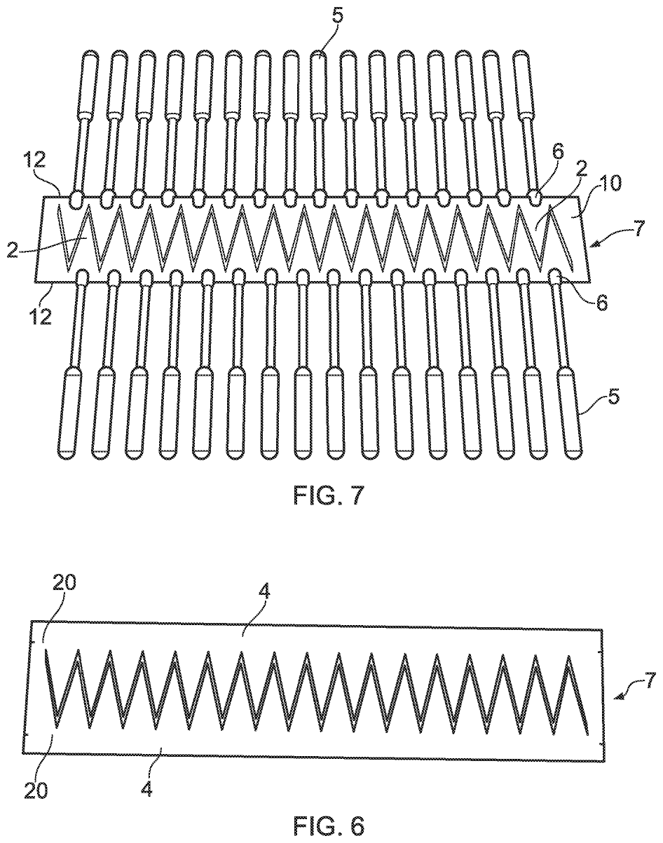

FIG. 6 shows how the sponge web 7 may optionally be trimmed along lines 20 after curing and pressing so as to adjust the width of the base portions 4. Although this will expose an additional cut surface 12, this cut surface will be held in the grip 6 of the handle portion 5 and hence will not come into contact with the surgical site. Accordingly, the risk of particulate contamination remains very low.

FIG. 7 shows the pressed sponge web 7 with handle portions 5 injection moulded and attached along both opposed edges 12 of the web 7.

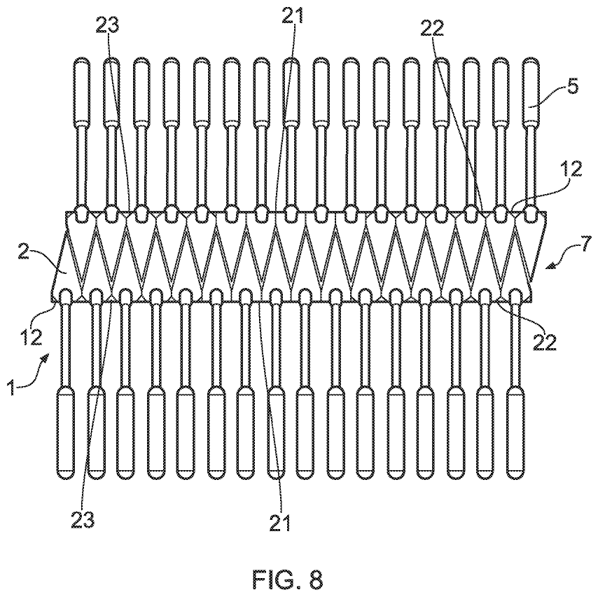

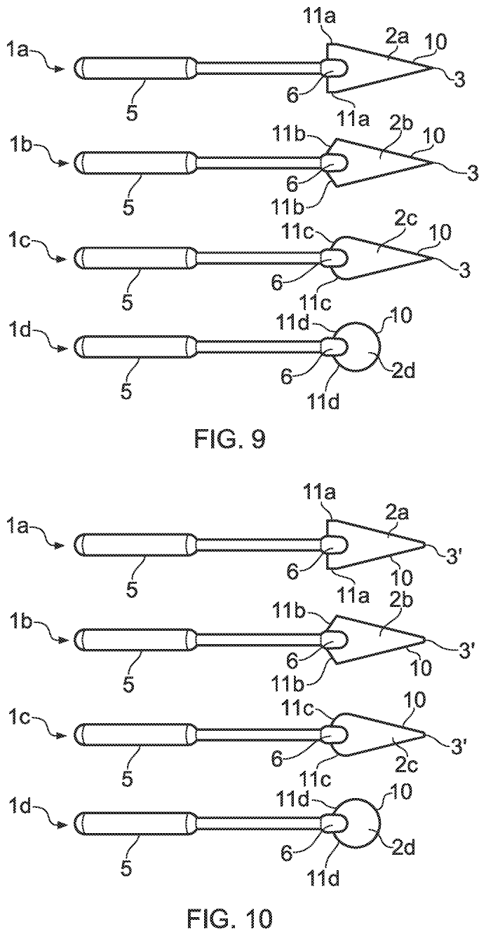

FIG. 8 shows how the pressed sponge web 7 with attached handle portions 5 may be cut into individual surgical spears 1 with sponge portions 2 of different shapes, as shown in FIGS. 9 and 10. Cut lines 21 are substantially perpendicular to a longitudinal axis of the sponge web 7 and result in surgical spears 1a with sponge portions 2a having parallel cut surfaces 11a. Cut lines 22 are straight but angled relative to the longitudinal axis of the sponge web 7, resulting in surgical spears 1b with coffin-shaped sponge portions 2b having convergent cut surfaces 11b. Cut lines 23 are curved, resulting in surgical spears 1 c with teardrop-shaped sponge portions 2c having curved cut surfaces 11c. It can be seen that the sponge portions 2a, 2b and 2c shown in FIG. 10 all have flattened apexes 3′, in contrast to the pointed apexes 3 of the sponge portions 2a, 2b, 2c of FIG. 9. This is simply to illustrate that different shapes may be obtained by appropriate configuration of the mould 15 and central divider 16.

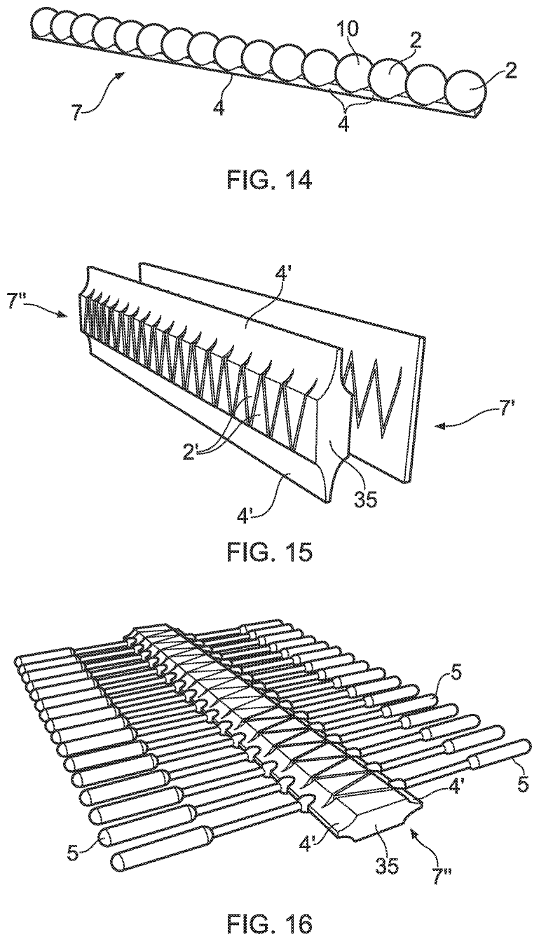

The surgical spear 1d of FIGS. 9 and 10 has a rounded sponge portion 2d with a rounded cut surface 11d. The rounded sponge portion 2d may be generally disc-shaped when pressed, and may expand into a ball-shaped sponge when subsequently wetted. The surgical spear 1d of FIGS. 9 and 10 may be made from a sponge web 7 as shown in FIG. 14.

Each of the sponge portions 2a, 2b, 2c and 2d has an uncut external surface 10 extending over substantially all of the external surface other than areas close to the grips 6 of the handle portions 5.

FIG. 11 shows an alternative sponge web 7 with sponge portions 2 having a conical shape, and connected to each other at base portions 4.

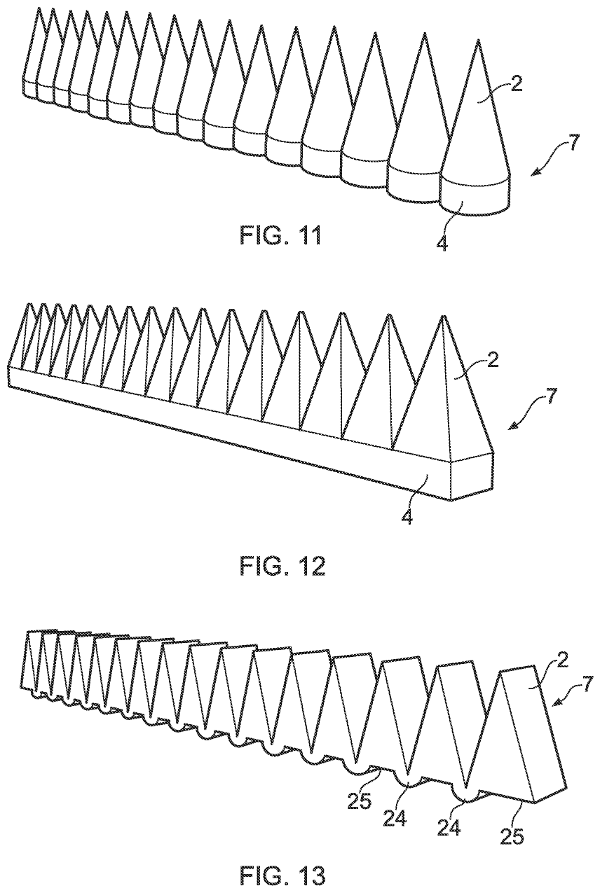

FIG. 12 shows an alternative sponge web 7 with sponge portions 2 having a pyramidal shape, and connected to each other at base portions 4.

FIG. 13 shows an alternative sponge web 7 with sponge portions 2 having a triangular prism shape, and connected to each other by thin bridging portions 24. It will be appreciated that in this embodiment, the exposed base surface 25 of each sponge portion can be formed as an uncut external surface, with only very small cut surface portions being exposed when cutting away the bridging portions 24 after attaching the handle portions 5.

FIG. 14 shows an alternative sponge web 7 with sponge portions 2 having a ball or spherical shape, and connected to each other at base portions 4. This sponge web 7 may be used for manufacturing the surgical spears 1d of FIG. 10.

In each of FIGS. 11 to 14, the sponge web 7 is shown in an expanded condition, for example before pressing. It is a property of certain open cell foam materials, in particular PVA sponge and cellulose sponge materials, that they may be cast in a mould, optionally washed or treated with chemical solutions, and then dried and pressed so as to occupy a much smaller volume. The dried and pressed material will be relatively hard and stiff. When the dried and pressed open cell foam material is wetted again, it will absorb liquid and expand to its original volume. In addition, the wetted, expanded open cell foam material may be significantly softer and more pliable than when in the dried and pressed form. Moreover, the soft, pliable, wetted and expanded open cell foam material, if allowed to dry will remain in the expanded state, although it will revert back to a relatively hard and stiff state.

Pressing the sponge web 7 also facilitates the attachment of the gripping members 6 of the handle portion 5 to the sponge portion 2.

FIG. 15 shows an alternative, partially pressed sponge web 7″ next to a fully pressed sponge web 7′. The partially pressed sponge web 7″ is pressed only along the base edge portions 4′, leaving a central portion 35 of the sponge web 7″ and the sponge portions 2′ in an expanded state. This may be preferred for certain applications.

As shown in FIG. 16, the partially pressed sponge web 7″ may have handle portions 5 attached to the pressed base edge portions 4′ while leaving a central portion 35 of the sponge web 7″ and the sponge portions 2′ in an expanded state. The sponge web 7″ can then be cut between the points of attachment of the handle portions 5 so as to form individual surgical spears 1.

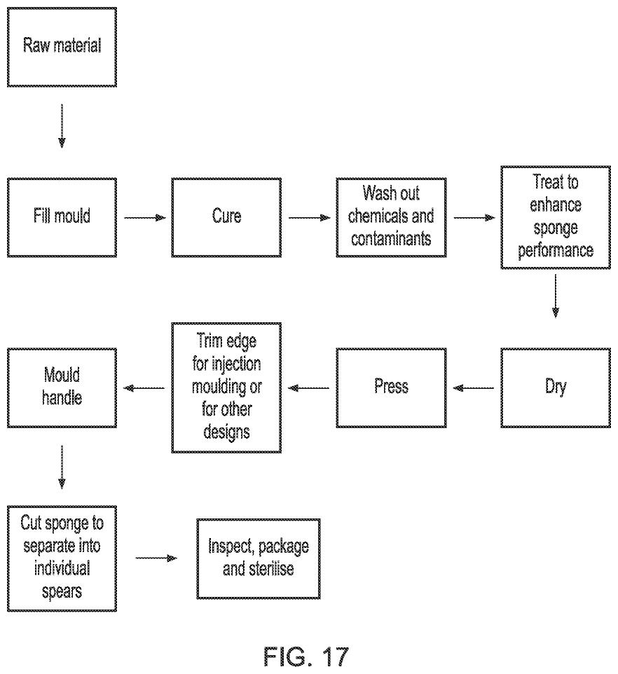

FIG. 17 is a process flow chart showing the manufacturing steps according to one embodiment. Firstly, an appropriate open cell foam forming composition in liquid form (e.g. PVA or cellulose-based) is prepared and then cast into a mould. The foam forming composition is allowed to react and cure in the mould, and the sponge web thus formed is then removed from the mould, washed, and chemically processed so as to remove any harsh chemicals or materials that were used to form and/or cure the foam material of the sponge web. An optional step of treating the sponge material to enhance its performance may be applied, an exemplary treatment formula being a mixture of surfactant and glycol. The web is then dried, pressed flat and the handle portions are moulded on a separate production line. The web is then separated by cutting into individual surgical spears, depending on the final shape required, with attached handles. Finally, the surgical spears are inspected, packaged and sterilised.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A surgical sponge device comprising:
a sponge portion and
a handle portion,
wherein the sponge portion is formed from an open cell foam material having an external surface, wherein the handle portion comprises a grip at one end thereof that mechanically grips mutually opposed external surface regions of the sponge portion an edge of the sponge portion, and
wherein substantially all of the external surface of the sponge portion is formed as an uncut external surface, except for at least one predetermined region of the external surface that is located close to the grip, the at least one predetermined region of the external surface being formed as a cut external surface.

2. The device as claimed in claim 1, wherein the uncut external surface is substantially free of particulate matter.

3. The device as claimed in claim 1, wherein the grip comprises a pair of mutually opposed gripping members.

4. The device as claimed in claim 1, wherein the sponge portion is formed from a polyvinyl alcohol-based (PVA) sponge material.

5. The device as claimed in claim 4, wherein the uncut external surface is less porous than the open cell structure inside the foam material.

6. The device as claimed in claim 1, wherein the sponge portion is formed from a cellulose-based sponge material.

7. The device as claimed in claim 1, wherein the sponge portion is formed from a polyurethane-based sponge material.

8. The device as claimed in claim 1, wherein the sponge portion is formed from a sponge material that is compressible when dry.

9. The device as claimed in claim 1, wherein the sponge portion has a substantially triangular or triangular prism shape, with an apex of the substantially triangular or triangular prism shape being disposed away from the handle portion, and an opposed base of the substantially triangular or triangular prism shape being gripped by the grip.

10. The device as claimed in claim 1, wherein the sponge portion has a substantially coffin-like shape.

11. The device as claimed in claim 1, wherein the sponge portion has a substantially teardrop-like shape.

12. The device as claimed in claim 1, wherein the sponge portion is compressed and relatively rigid prior to use, and wherein the sponge portion expands and softens upon absorption of liquid.

13. The device as claimed in claim 1, wherein only an edge region of the sponge portion that is held by the grip is compressed, the rest of the sponge portion being in an expanded configuration even when dry.

14. A sponge web formed from an open cell foam material, the sponge web comprising:
a plurality of individual sponge portions each having a base edge and an opposed tip region, the sponge web having an uncut external surface, and adjacent sponge portions being connected to each other in the web at their base edges.

15. The sponge web as claimed in claim 14, wherein the uncut external surface is substantially free of particulate matter.

16. The sponge web as claimed in claim 14, wherein the plurality of individual sponge portions comprise first and second rows of facing sponge portions arranged such that the respective tip regions of the first and second rows interdigitate but are spaced from each other by a gap.

17. The sponge web as claimed in claim 14, further comprising a plurality of handle portions each having a grip at one end thereof, the grip of each handle portion being attached to the base edge of an individual sponge portion.

18. The sponge web as claimed in claim 14, formed from a polyvinyl alcohol-based (PVA) sponge material.

19. The sponge web as claimed in claim 18, wherein the uncut external surface is less porous than the open cell structure inside the foam material.

20. The sponge web as claimed in claim 14, formed from a cellulose-based sponge material.

21. The sponge web as claimed in claim 14, formed from a polyurethane-based sponge material.

22. The sponge web as claimed in claim 14, formed from a sponge material that is compressible when dry.

23. A method of manufacturing a surgical sponge device comprising a sponge portion and a handle portion, the method including the steps of:
i) providing a mould configured to define a plurality of voids that are connected to each other only along at least one edge portion of the mould;
ii) filling the mould with reagents selected to generate an open cell foam material within the mould, the open cell foam material taking the form of a plurality of sponge portions having shapes defined by the plurality of voids, the sponge portions connected to each other only at base regions thereof corresponding to the void connections in the mould, the open cell foam material having an uncut external surface where it contacts the mould;
iii) removing the open cell foam material from the mould;
iv) washing and treating the open cell foam material;
v) subsequent to step vi); connecting multiple handle portions to the open cell foam material at the base regions of the sponge portions and cutting the open cell foam material to separate the open cell foam material into a plurality of surgical sponge devices, each device having a handle portion connected to a sponge portion formed of the open cell foam material having the uncut external surface formed substantially all of its external surface except for at least one predetermined region of the external surface that is located close to the point of connection of the handle portion, the at least one predetermined region of the external surface being formed as a cut external surface, and wherein the handle portions each comprise a grip at one end thereof that mechanically grips mutually opposed external surface regions of the sponge portion an edge of the sponge portion.

24. The method according to claim 23, wherein the mould comprises a single mould cavity with a plurality of voids, each void defining a sponge portion.

25. The method according to claim 23, wherein the mould has a saw-tooth configuration, each void comprising a tooth of the saw-tooth configuration.

26. The method according to claim 23, wherein the mould includes a central divider having a zig-zag or wave profile defining two sets of complementary, inter-engaging voids.

27. The method according to claim 23, wherein the open cell foam material is dried and pressed between steps iv) and v).

28. The method according to claim 27, wherein only edge portions of the open cell foam material are pressed between steps iv) and v).

29. The method according to claim 23, wherein inside surfaces of the mould have a smooth texture, thus resulting in an external skin on the open cell foam material that has a smooth texture.

30. The method according to claim 23, wherein inside surfaces of the mould have a roughened texture, thus resulting in an external skin on the open cell foam material that has a roughened texture.

31.-33. (canceled)
